(19) United States
(12) Patent Application Publication
(10) Pub. No.: US 2015/0305630 A1
(43) Pub. Date: Oct. 29, 2015

(54) System and method for determining and monitoring a body system state

(71) Applicant: V. A. Shiva Ayyadurai, Cambridge, MA (US)

(72) Inventor: V. A. Shiva Ayyadurai, Cambridge, MA (US)

(21) Appl. No.: 14/120,114

(22) Filed: Apr. 25, 2014

(51) Int. Cl. A61B 5/0205; A61B 5/00; A61B 5/145

(52) U.S. Cl. CPC A61B 5/0205; A61B 5/14546; A61B 5/14507; A61B 5/742; A61B 5/0022; A61B 5/021

(57) ABSTRACT

This invention provides a system and method for determining a user's Current System State so as to enhance balance and well-being. A determination process computes force vector values based upon questions and integration of other biological measurements provided via a user interface. These force vector values can include factors such as Transport, Conversion and Storage. A display generator creates a representation of a location of Current System State based upon the force vector values versus a location for Natural System State. The representation defines a three-dimensional volume and the force vector values are arranged in three-dimensions. The user's Natural System State is located at a location in the volume and the Current System State is at another location. The force vector values are each illustratively represented as a discrete color spectrum. Users access the system through a client interconnected through the Internet.

### FIELD OF THE INVENTION

This invention relates to systems and methods for determining and monitoring personal well-being.

### BACKGROUND OF THE INVENTION

Personal well-being in the form of mental, spiritual and physical health are keys to a fulfilling life. Many factors affect well-being, including genetics, environment, personal, relationships, work, food and medications. People may strive for, but few actually achieve the proper balance of factors to maximize well-being.

### SUMMARY OF THE INVENTION

This invention overcomes disadvantages of the prior art by providing a system and method for determining a user's Current System State so as to enhance balance and well-being. A determination process computes force vector values based upon a combination of one or more pluralities of questions and biological measurements, provided via a user interface. These force vector values can include factors such as Transport, Conversion and Storage. A display generator creates a representation of a location of Current System State based upon the force vector values versus a location for one's Natural System State. The representation illustratively defines a three-dimensional volume and the force vector values are arranged in three-dimensions therein. The user's Natural System State is located at a particular location in this three-dimensional space, and the Current System State is located at another particular location in this three-dimensional space, representing displacement from the Natural Systems State. The force vector values are each illustratively represented as a discrete color spectrum. Illustratively, users access the determination process through a client computing device and display interconnected through the Internet. A patient/client (user) can also be served by a practitioner who employs a portal to answer various queries/questions related to the user based upon the user's prior or contemporaneous oral/written answers (and other information—such as biological parameters).

### BRIEF DESCRIPTION OF THE DRAWINGS

The invention description below refers to the accompanying drawings, of which:

FIG. 1 is a diagram representation a three-dimensional vector space/spectrum in which a person's natural body constitution (Natural System State e.g. using the red dot) as well as the person's current situation (Current System State e.g. using the black dot) can be represented;

FIG. 2 is a schematic diagram showing a networked system for inputting Natural System State-related information from web-based clients to a server arrangement that performs a Natural System State determination process;

FIG. 3 is a diagram of a user interface screen providing a series of questions/queries dealing with Transport for use in the determination process, and establishing a user's Natural System State;

FIG. 4 is a diagram of a user interface screen providing a series of questions/queries dealing with Conversion for use in the determination process, and establishing a user's Natural System State;

FIG. 5 is a diagram of a user interface screen providing a series of questions/queries dealing with Storage for use in the determination process, and establishing a user's Natural System State;

FIG. 6 is a diagram of a user interface screen providing an explanation of Natural System State in connection with the spectrum diagram of FIG. 1;

FIG. 7 is a diagram of a user interface screen providing a starting point for a user to determine his or her Current System State;

FIG. 8 is a diagram of a user interface screen providing residence queries to a user in connection with the determination of Current System State;

FIG. 9 is a diagram of a user interface screen providing a series of questions/queries dealing with Transport for use in the determination process for establishing a user's Current System State;

FIG. 10 is a diagram of a user interface screen providing a series of questions/queries dealing with Conversion for use in the determination process for establishing a user's Current System State;

FIG. 11 is a diagram of a user interface screen providing a series of questions/queries dealing with Storage for use in the determination process for establishing a user's Current System State;

FIG. 12 is a diagram of an exemplary run of the determination algorithm/process on user-entered data;

FIG. 13 is a diagram of a user interface screen depicting the user's Current System State on the spectrum representation and detailing the stability of the user's Transport, Conversion and Storage vectors;

FIG. 14 is a diagram of an interface screen enabling a user to subscribe to a continuing (premium) version of the system to thereby allow periodic monitoring of current system state;

FIG. 15 is a diagram of an interface screen enabling a practitioner or other interested third party to subscribe to a continuing (premium) version of the system to thereby allow periodic monitoring of current system state of a patient/client;

FIG. 16 is a diagram of a user interface screen containing recommendations for stabilization of a user's Transport vector;

FIG. 17 is a diagram of a user interface screen containing recommendations for stabilization of a user's Conversion vector;

FIG. 18 is a diagram of a user interface screen containing recommendations for stabilization of a user's Storage vector;

FIG. 19 is a diagram of a daily plan that is received by a user via e-mail in an effort to stabilize to a Natural System State;

FIG. 20 is a diagram of an optional user interface screen allowing a user to upload biological parameters and information for use in the determination and monitoring process according to an embodiment;

FIG. 21 is a diagram of an optional user interface screen presenting dialog boxes for entry of a plurality of specific biological parameters and/or other related information relevant to a user according to an embodiment;

FIG. 22 is a diagram of an optional user interface screen enabling a user to upload entered biological parameters and/or other information provided in accordance with FIG. 21 in a desired computer file format;

FIG. 23 is a diagram of an optional user interface screen presenting results for the user's Transport, Conversion and Storage vectors in view of uploaded biological parameters;

FIG. 24 is a diagram of an optional user interface screen that contains the link to a portal (“Practitioner Portal”) that provides third party practitioners with access to the illustrative system and method for use in analyzing system state of a patient/client;

FIG. 25 is a diagram of a user interface screen containing the home page for the Practitioner Portal with a box that provides brief instructions on getting started with the portal

FIG. 26 is a diagram of a user interface screen for the Practitioner Portal, containing a practitioner registration/log-in dialog;

FIG. 27 is a diagram of a user interface screen for the Practitioner Portal, for use by the previously registered practitioner in registering a client/patient (user) for whom an analysis is to be performed;

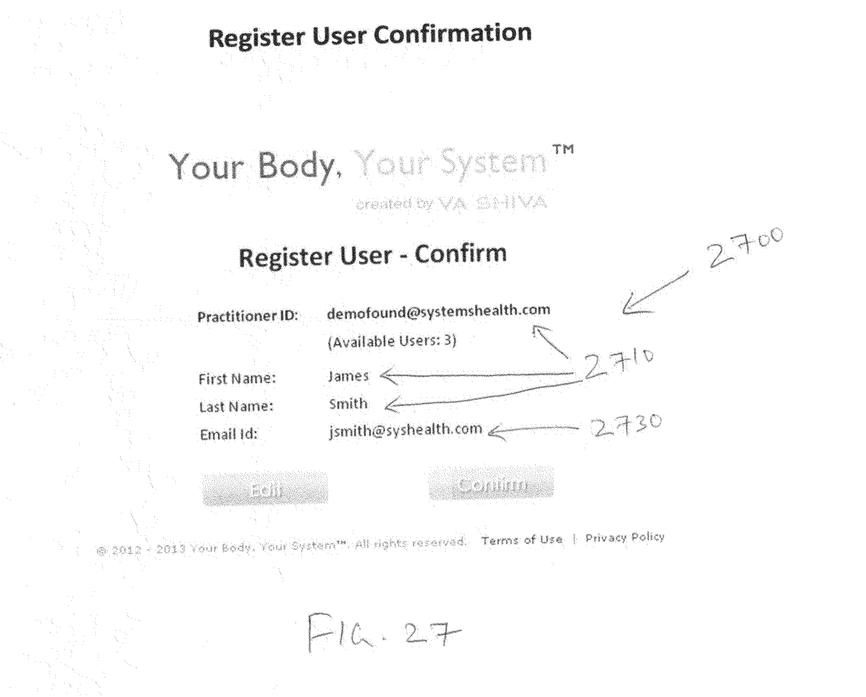

FIG. 28 is a diagram of a user interface screen for the Practitioner Portal allowing the practitioner to enter the system for performing an analysis (check-up) on a previously registered client/patient (user); and

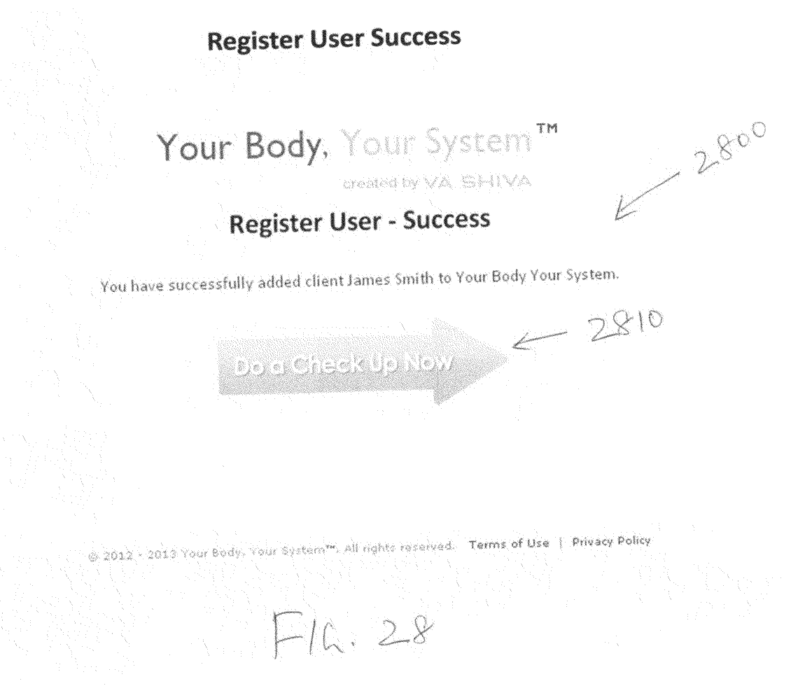

FIG. 29 is a diagram of an exemplary user interface screen for the Practitioner Portal of FIG. 24, providing a series of questions/queries dealing with Transport for use in the determination process for establishing a user's Natural System State (with other above-described queries and/or biological parameter-entry screens being similarly presented by the portal).

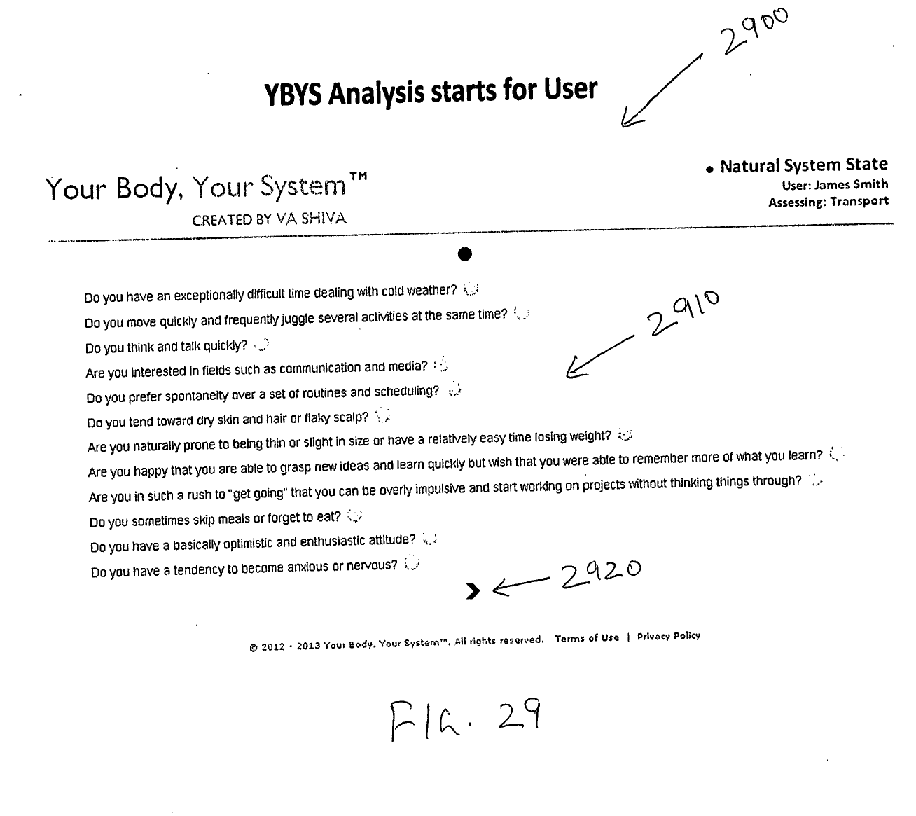

### DETAILED DESCRIPTION

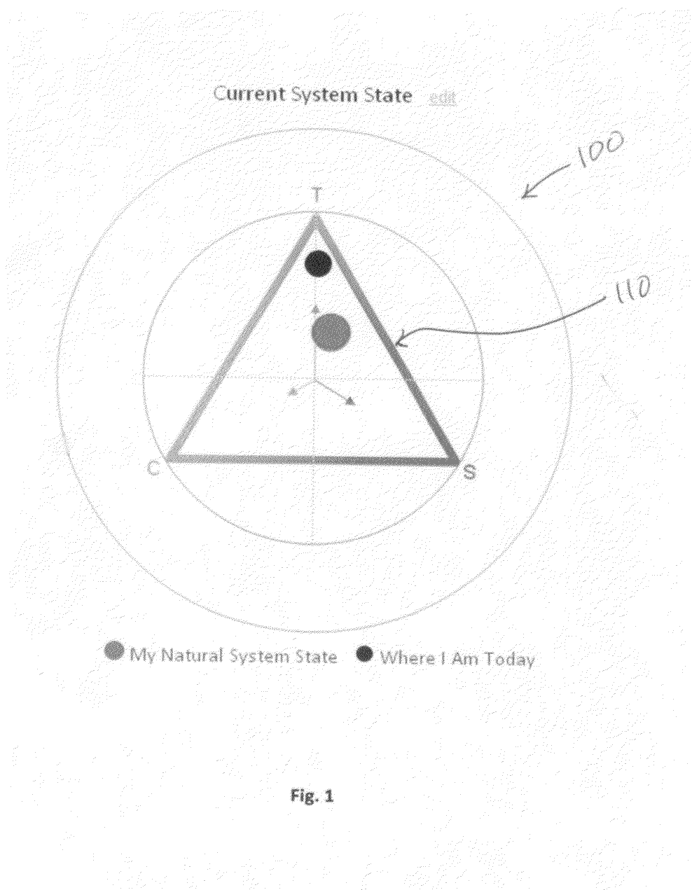

FIG. 1 is a diagram 100 of a representation of a person's Current System State versus an (optimal) Natural System State. For the purposes of this description, a system, method and process for determining and monitoring system state can be also termed by the proprietary trademark “Your Body, Your System™” (YBYS). More generally, illustrative embodiments herein provide a computer software-based process, algorithm, tool and website that maps a person's natural body constitution (Natural System State) and current body constitution (Current System State) state to a three-dimensional space (as shown in the diagram 100, and provides recommendations of dietary/exercise/lifestyle changes to balance the person's Current System State with the Natural System State.

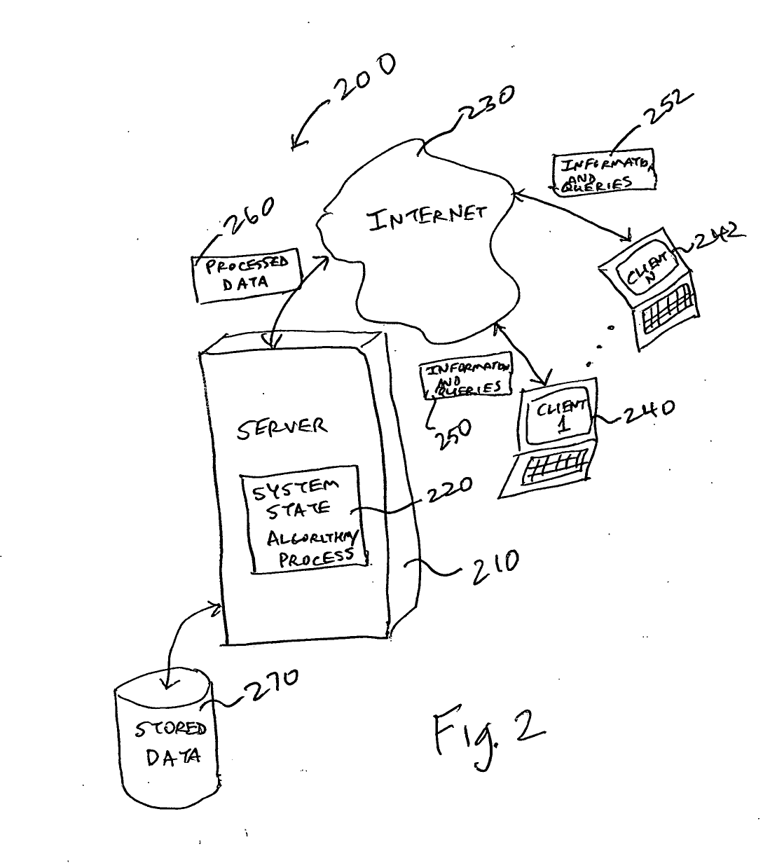

With further reference to FIG. 2, the system is deployed within a server environment 200 that can include one or more servers (or any acceptable non-transitory software-processing/computing) device(s) 210 that can include an instantiation of the system software and related algorithms/processes 220. As used herein the terms “process” and/or “processor” should be taken broadly to include a variety of electronic hardware and/or software based functions and components. Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor herein can be implemented using electronic hardware, software consisting of a non-transitory computer-readable medium of program instructions, or a combination of hardware and software.

The server arrangement 210 communicates over a network 230, such as the public Internet, and interconnects various client devices 240, 242, including, but not limited to PCs, cellular phones, laptop computers, tablet computers, and the like. These client devices are employed by users of the system to exchange information and queries 250, 252. The devices can be equipped with an appropriate browser that supports a user interface arrangement provided by the system. The server arrangement 210 receives client information (250, 252), and returns processed data 260 based upon client inputs and the outcome of the algorithm/process. Data for use with the algorithm and related to clients is stored in an appropriate storage arrangement 270. All processing and communication components can be implemented in accordance with ordinary skill using commercially available devices.

In further summary, and with reference again to FIG. 1, YBYS provides of a user interface where the user is asked a series of questions (queries) pertaining to the balance of three essential forces in the body—Transport (T), Conversion (C) and Storage (S). These questions can also be integrated with other biological measurements. The responses and measurements are then mapped to a three dimensional spectrum 110, and using a force vector algorithm, the person's Natural System State is established as a point within the spectrum.

By way of further illustration, the vectors of Transport, Conversion and Storage in association with Natural and Current System State can be further explained as follows:

Transport

Users who are dominated by the vector of Transport are highly sensitive to variations in flow, mobility, and movement and will have issues when these elements are not functioning properly. If the forces of Transport are out of sync, one can experience a wide variety of health problems. If one is dominated by this force, one needs to notice when such Transport forces are not optimal. If such forces are too high, for example, then one can become overly hyperactive, and thus it is desirable to monitor Transport and attempt to optimize it to avoid undesirable conditions like hyperactivity.

Transport is responsible for the movement of energy and information, as well as matter; this includes the flow of input and output across all levels of the human system. Therefore, Transport is in charge of everything that moves—everything that is kinetic. Within the human body, this includes walking as well as breathing, elimination as well as blood flow, communication (movement of information) skills as well as the body's central nervous system. Transport moves all five elements—air, space, earth, water, and fire. Because of this, of all the forces, Transport can be regarded as the “king of the hill,” or leader. Without the force of Transport, then operation of the forces of Conversion or Storage (described further below) could be highly problematic. When Transport is not functioning correctly within the body, then all the other forces can become unbalanced rather quickly.

By way of further example, when a man or woman, whose system is dominated by Transport, is resonating, the results can be highly desirable for the user. One exemplary user with dominating Transport forces is able to communicate well, using his extraordinary verbal skills. Another exemplary user, who also has dominating Transport forces, is usually the most enthusiastic person in the room. These exemplary users both move very quickly and energetically, regardless of their activities. This is an example of Transport functioning optimally, at its peak. Transport forces help one grasp the overview and provide the skill set to speedily understand what's required. Thus, someone with Transport dominance is typically a quick learner (they literally transport information from their environment into themselves). But when TRANSPORT forces become unbalanced, there can be some destabilizing situations. The person driven by Transport can experience difficulty with details and can sometimes find it difficult to retain needed information. Transport forces can also create problems with process. For example a person dominated by Transport might know how to drive and have no problem passing the test, but is stymied by the orderly steps required to get to the place where he could even be tested. The executive functioning tasks of making the phone calls, obtaining forms, filling out the forms, standing in line, being on time for the test appointment, etc., is all too daunting for such a person, despite his clear intelligence. Note that the forces of Transport are composed of air and space. Translated into the human realm, this means that all those dominated by Transport need to be careful that he or she does not become so involved in the airy world of ideas that they lose their connection to earthier elements.

Conversion

A person dominated by the force of Conversion is typically highly sensitive to variations in transformational processes such as metabolism, digestion, conversion of ideas, thinking analytically and making clear decisions. If the forces of Conversion are not functioning well, one can experience health and emotional problems associated with the inability to convert and transform elements of Matter, Energy and Information. Illustratively, fire is the element most closely associated with the forces of CONVERSION. The forces of Conversion, like those of fire, have the power to transform matter, energy, and information, converting one element to another. Conversion is what takes place during the process of transformation. Like a knife, which can cut and transform, Conversion is related to the qualities of sharpness as well as heat. Conversion is most closely associated with those systems in Nature that are like “engines” and able to take in one form and convert it to another. Notably, engines generally operate well in a cooling environment, like a car's engine, which takes in chemical energy (gasoline) and converts it to mechanical energy (rotational movement of the wheels). Typically, this engine requires a radiator (or similar heat-transfer device) to cool itself—because engines do not operate well in an overheated environment. The processor on a computer known as the Central Processing Unit (CPU) is dominated exclusively by Conversion. It obtains symbols, and calculates and converts those input symbols to other symbols. The CPU is almost always accompanied by a heat-transfer device, such as a fan, which is cooling the CPU to ensure it does not become overheated.

One who is dominated by Conversion can dislike the summer months, and prefer cool temperatures. Those dominated by this force seem to generate their own heat source. Those who are Conversion dominant often appear to have a furnace-like capacity to give off heat. Men and women, who are driven by Conversion, tend to have fiery personalities or attitudes. When all is going well with their nature, these forces assist Conversion-dominant people in maintaining generally sweet and happy dispositions, subject to the natural pitfalls of life. Some people dominated by Conversion manage to keep their “fire” hidden, but they are always determined, no matter what the circumstances. They might be more infrequently sharp-tempered, but they are typically strong willed. And, if they are out of balance, they tend to be overly argumentative. They like to cut through unnecessary conversation and want people to “get to the point.” They love efficiency, frequently consider themselves experts at it, and hate to waste time. This quality can make them impatient. They hate it when they don't get things right and can be as hard on themselves as they are on anyone else. And, when the forces of Conversion are imbalanced, their tempers can flare.

The forces of Conversion can provide people with significant ability in the skill of actualizing an idea to reality. Such people are natural entrepreneurs and tend to be ambitious and exceptionally good with work that is challenging and requires attention to detail. These men and women are attracted to fields like science and engineering. They are often also the consistent and valuable sales people in an organization, because they are good at enabling others to realize value in an idea, and persuade them to pay for it. They are also able to figure out practical and manageable solutions for complex problems. Conversion forces also fan one's competitive instincts, and, for example, attract people to debating teams where they can put their well-honed skills of arguing and detailed analysis to good use. In general, many Conversion-dominant people tend to hold onto a particular issue and are reluctant to “let it go.” Within the body, Conversion is responsible for metabolism. People dominated by this force usually have the ability to metabolize food, and tend to have huge appetites for food and drink. However, those dominated by Conversion forces need to be aware of the potential for digestive problems that can be caused by eating too much spicy foods—it is simply just too much “fire” for them. Conversion forces can also make one's skin and eyes redden and flare up. These forces support success in warrior-like environments such as competitive sports or commodity trading, but they need to be modulated when engaging in activities that “heat” them up, to the point where they run the risk of “burning up” or “burning out.”

Storage

Storage generally relates to structure, in that a body requires a structure to contain matter, information, and energy. To hold water, for example, a container such as a glass is used, providing appropriate storage. An organization generally requires structure in the form of policies, rules and guidelines, which holds the organization together. In the context of a living organism, its cells contain a cytoskeleton, which provides shape and structure to contain all the organelles in your cell. A body comprises a skeletal structure including bones, which provide the structure to contain it in a predefined shape and form.

Storage is a vector centered around structure. Those dominated by Storage rely on their structure (a body frame which is usually relatively substantial) for themselves, as well as providing structure for others. They can be very caring and are reliable helpers/partners. Storage is made up of the elements of earth and water. By way of illustration, earth and water mix to create bricks, which provide the foundation structure of any building. If the forces of Storage are not functioning well, that same structure can become one of inertia, a muddiness (of earth and water), that does not move, or moves very slowly, and can be depressed. Like the earth itself, men and women who are dominant in Storage are naturally able to “hold” more than the average person. Storage almost always requires those who are dominated by it to watch their weight. (Men tend to be Thickset, and women voluptuous.)

To understand more about Storage, think about what it means to provide structure and store things. The forces of structure typically drive people to be natural caretakers—father and mother earth types, genuinely concerned with the welfare of those they know. Storage dominant men and women seem naturally able to provide a strong structure and foundation for others to lean on. A Storage dominance is good for saving money, possessions and energy; it is bad for losing weight. If you are fortunate enough to have a friend who is dominated by this force, you know you have somebody with whom you can share your secrets assured that this person will likely store, hold, and protect whatever he/she is told.

A person who is dominated by Storage, can engage in significant external care taking of others, and not apply enough internal nurturing of himself or herself. In this regard, the person's friends may “dump” their problems on the Storage-dominant person's capable shoulders. Storage forces help a person stay relaxed, calm, capable, peaceful and a Storage-dominated person usually projects steadiness and an aura of security. But when a Storage-dominated person is out of balance, he or she can become stubborn or seem to be infused with an all-pervasive “dullness.” It can feel as though the world is moving way too fast and there appears to be no viable place to jump in and become part of it. Unlike the person dominated by Transport forces, where movement is the order of the day, the Storage forces tug one in the direction of retreat, stillness, and non-movement. Someone with strong Storage forces can sometimes feel inarticulate or be reluctant to “open up” or talk. A Storage-dominant individual tends to learn new information much more slowly than a person dominated by Transport forces, but the Storage person is able to retain and remember more of what has been learned. This is because such persons have the structure to store that information for long periods. Also, because of their natural ability to hold information, those with dominant Storage forces make very good managers providing structure, process, and organization. A good financial money manager exhibits strong Storage skills. Likewise, a highly valued assistant who is able to hold the facts about an employer's life, work, and business in his/her head is showing strong Storage forces. Many good psychologists, doctors, nurses, therapists and body workers are exhibiting Storage dominance by the manner in which they are for their patients and clients, as are Chief Operating Officers who are be able to hold and manage all aspects e.g. legal, product development, finance, etc. of their business, and define new processes and structures for making the vision of a CEO into reality by delivering products and profit. Physically, those with a dominant Storage nature tend to have issues with mucous, and sometimes have illness such as asthma, sinusitis, or frequent colds. They can also possess a tendency to become depressed.

In general, a client logs onto YBYS and is presented with a user interface via a browser screen. The browser screen explores both the user's Natural System State and then seeks to establish a Current System State as a comparison. Both of these values can be displayed as indicated on a three-dimensional vector space diagram 100. The user interface first seeks to establish the user's Natural System State by providing him or her with a series of queries (e.g. twelve questions) in each of at least three key vector forces as described above, namely, Transport, Conversion and Storage. The queries are specifically designed to provide appropriate date relevant to Natural System State for each vector. Later in the process, following establishment of the user's Natural System State, the interface then seeks to establish the person's Current System State through another set of questions and responses. These questions (e.g. ten per vector) are tailored to establishing the user's Current System State in each vector. As also described further below, the queries on Natural System State and Current System State can include the ability to integrate other biological measurements relevant to the user, such as Cholesterol, Heart Rate, various blood factors, etc. Using the same force vector algorithm for both Natural System State and Current System State, each value is provided as point within the diagram's vector space (also termed a “spectrum”) 100 as shown in FIG. 1.

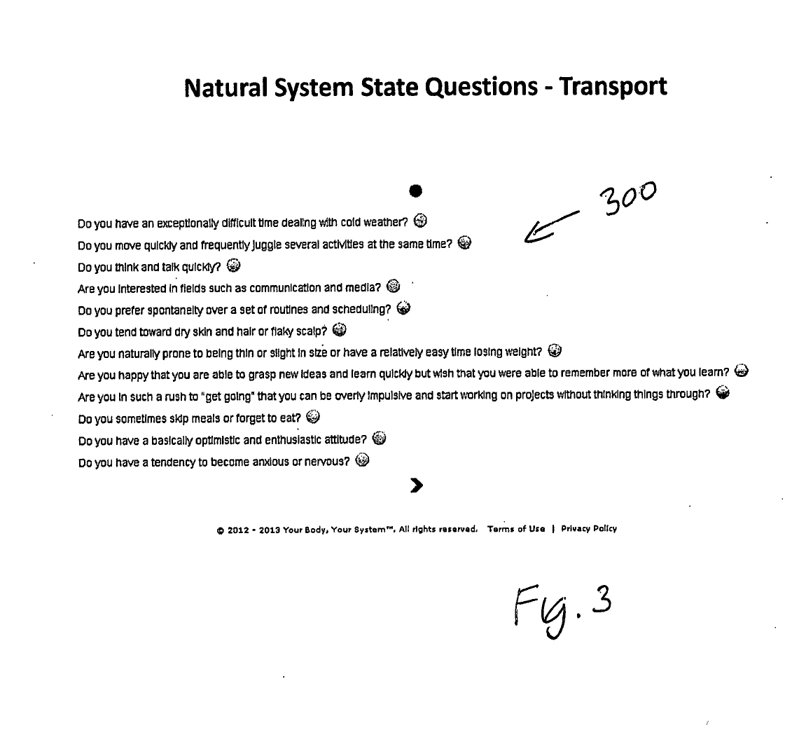

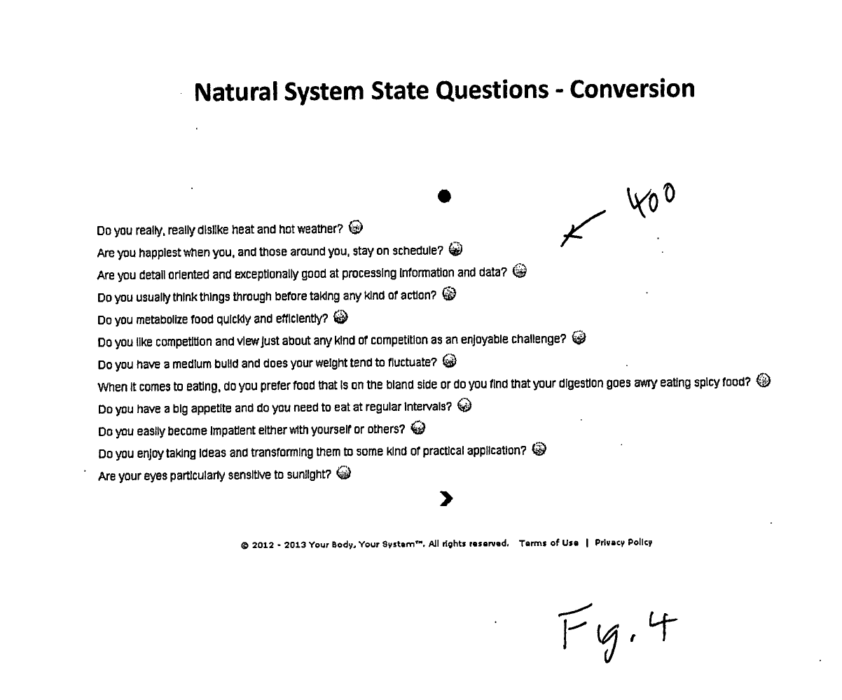

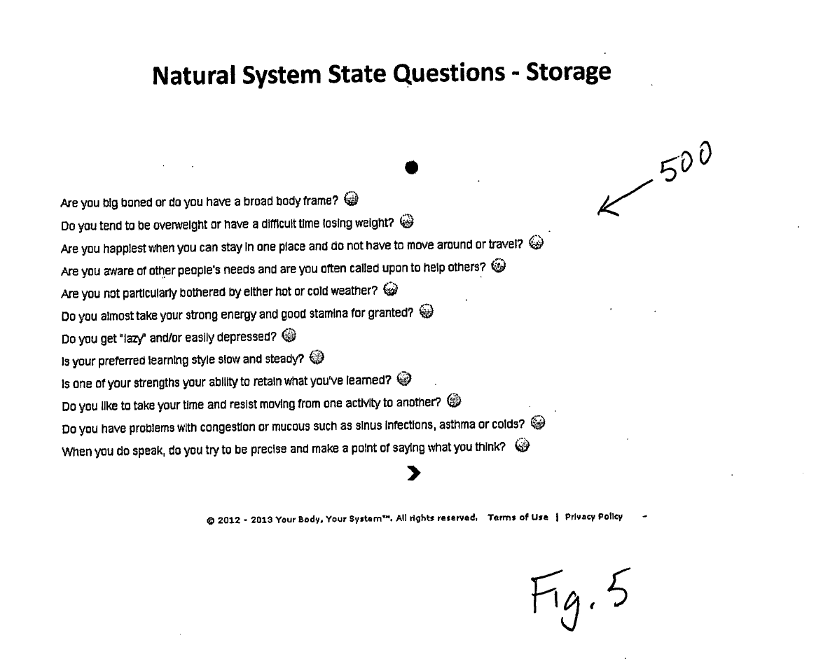

With respect to the particular queries provided by the interface, FIG. 3 details a screen 300 asking a series of questions dealing with Transport—the relevance of which to this vector should be self-explanatory. These questions are self-explanatory to the reader. Similarly, FIG. 4 details a screen 400 containing a series of self-explanatory questions related Conversion. FIG. 5, likewise, depicts a screen 500 containing self-explanatory questions dealing with Storage.

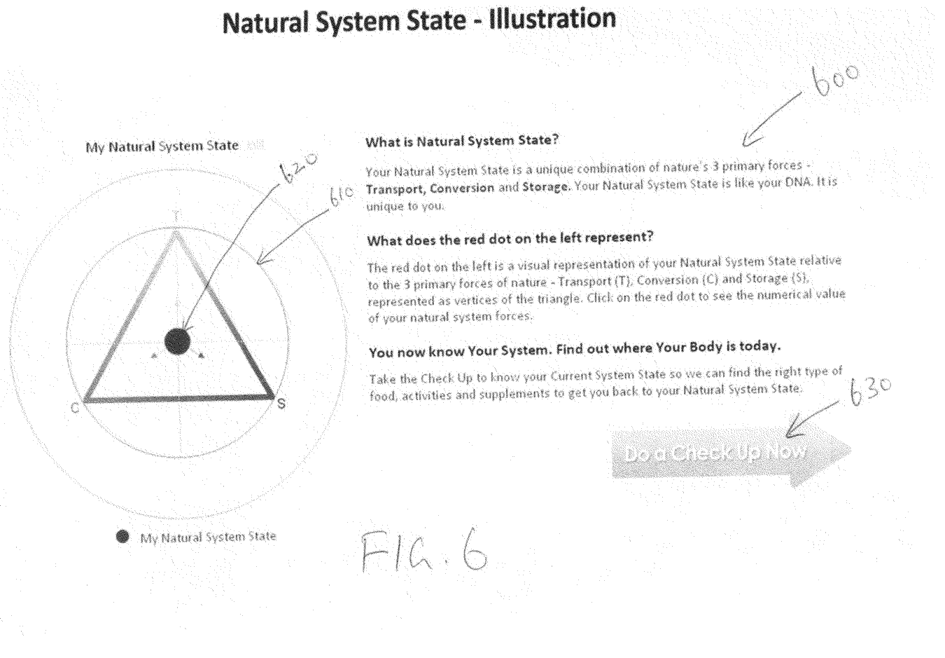

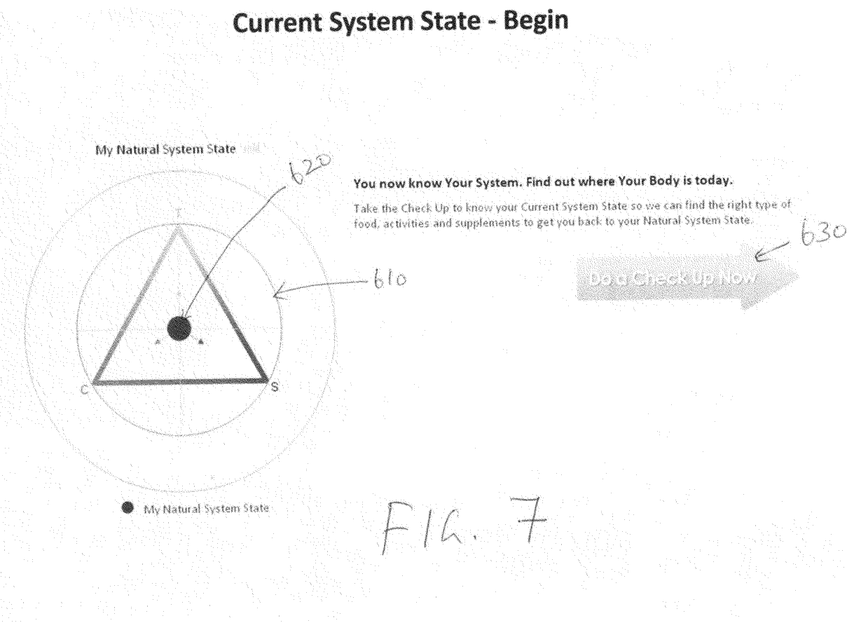

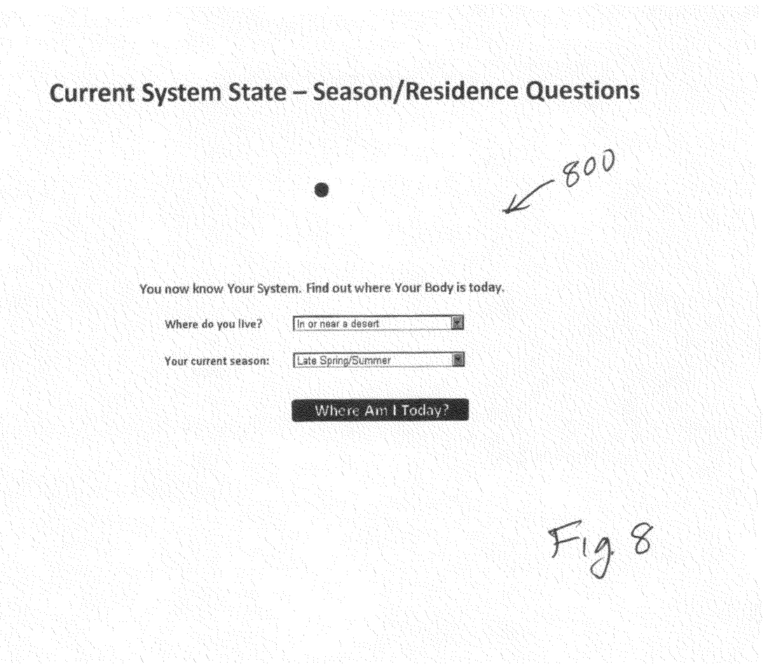

FIG. 6 is a self-explanatory interface screen 600 describing how the spectrum diagram 610 operates to represent the three vectors of Transport (T), Conversion (C) and Storage (S), making up the Natural System State. In general, the depicted (red) dot 620 locates the user's Natural System State on the diagram 610. It need not be centered within the represented three-dimensional space as each person's Natural System State can vary from a centered location within the diagram. The red dot can be clicked upon with a cursor to access a numerical value display of natural system force vectors. This display includes a start button 630, that the user can click to access the first screen 700, FIG. 7 used by the determination process in computing the user's Current System State. This screen 700 leads to the initial query screen 800 in FIG. 8. This screen includes questions about type of location in which the user resides and the season. Note that these functions, and other selected data-entry functions can be automated (occurring free of user-input) where the user's browser (or an application, such as Google) already contains data on location, time of year, etc.

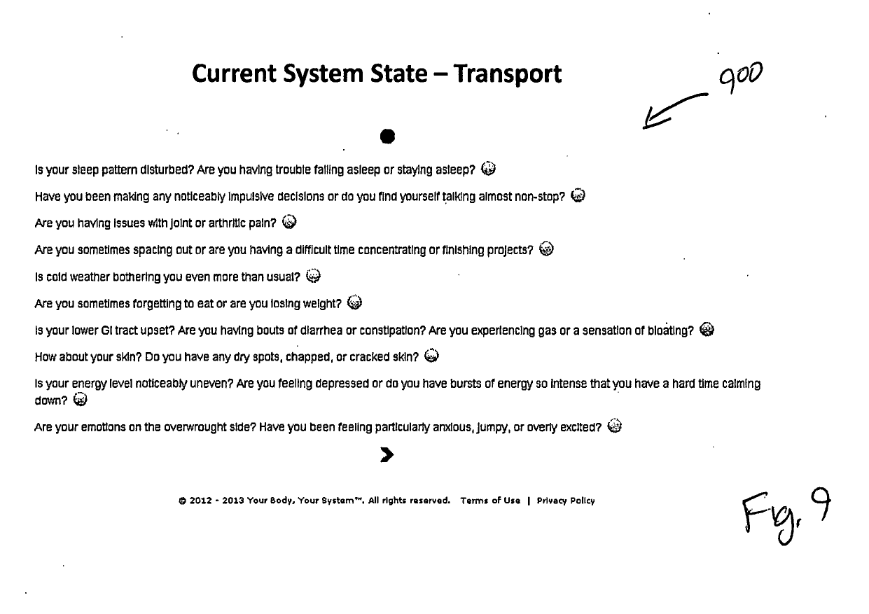

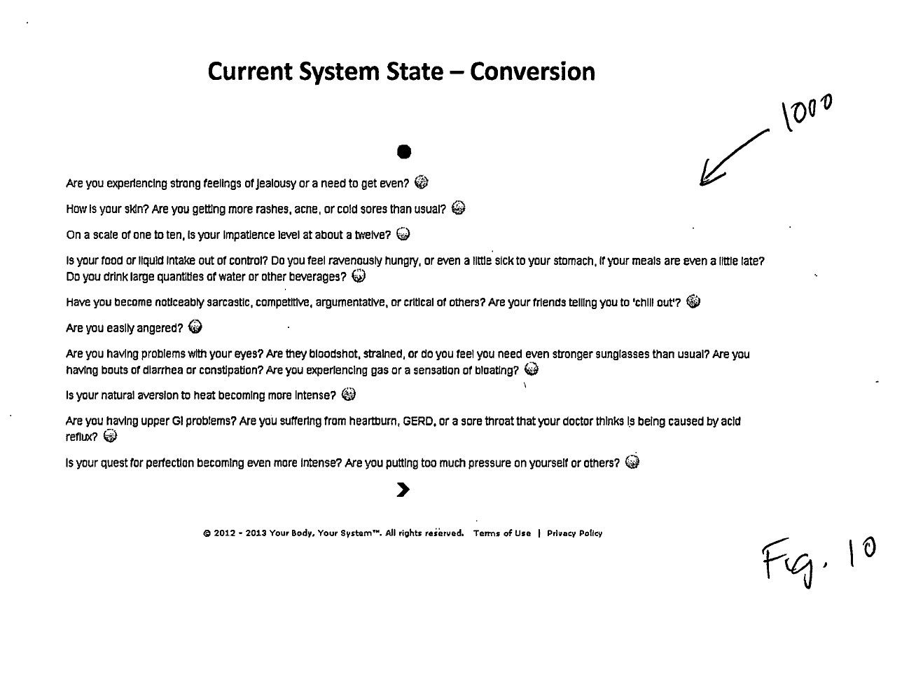

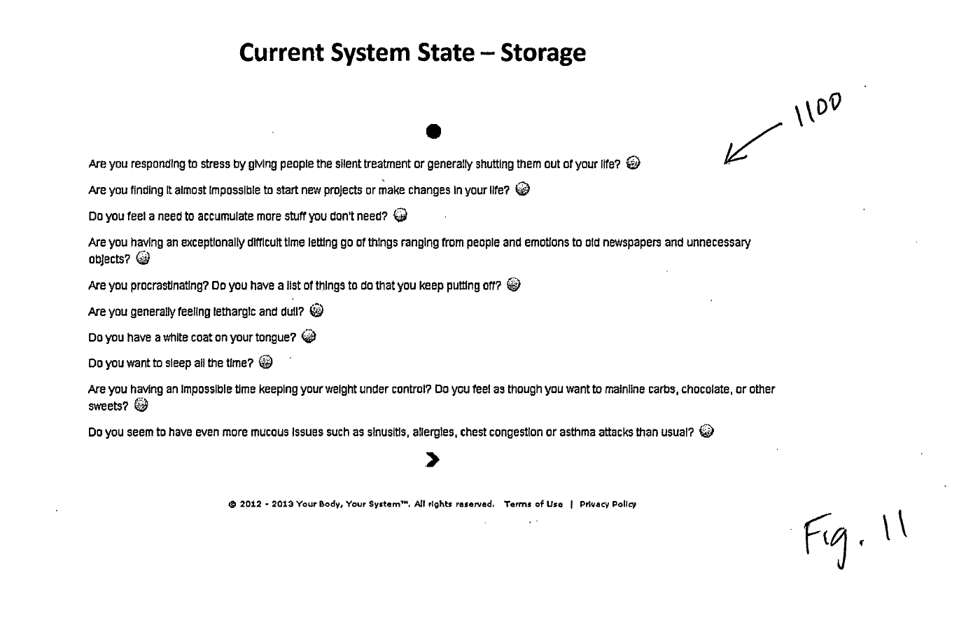

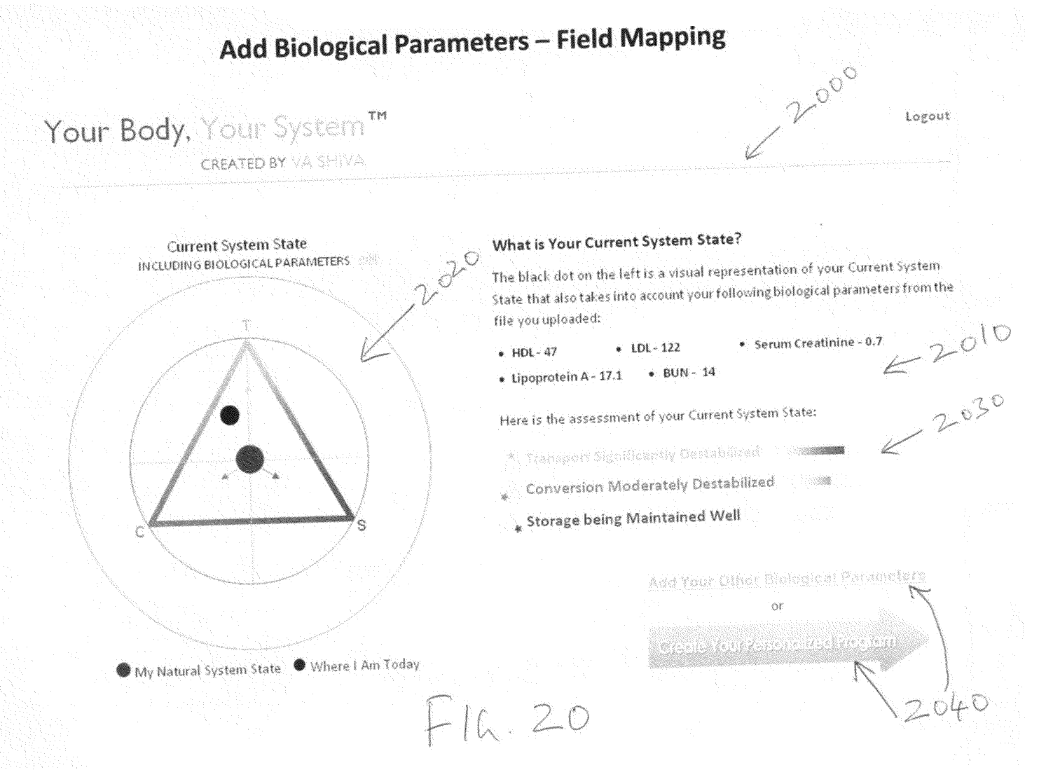

After answering the queries on the screen 800 of FIG. 8, the system exposes the screen 900 of FIG. 9. This screen asks the user a series of self-explanatory questions, similar in scope to those asked during the Natural System State determination stage (FIGS. 3-5). Screen 900 queries Transport-based issues. Screens 1000 and 1100 (FIGS. 10 and 11), respectively query Conversion and Storage-based issues. All questions are directed to determine the dominance of each vector as it is generally characterized above. Illustratively, queries for both Natural System State and Current System State determination are of a yes/no variety, but can have a multiple-choice or quantitative response in alternate embodiments. By way of example, questions can further include various biological measurements, including, but not limited to, ethnicity, height, weight, blood type, blood pressure reading, cholesterol count, hormone levels (and/or other metrics typically obtained in a lab analysis of blood and/or other fluids), urinalysis, body mass index, and the like. This is described further below with reference to FIGS. 20-23.

The answers to the various queries are then stored and used by the system determination process (algorithm) to generate the three System State force vectors for Transport, Conversion and Storage. The first set of 12 (illustratively) questions is relevant to the force of Transport. Answering “Yes” to each of these questions assigns a value of 1 to the answer. Not answering, or answering “No” is assigned a value of 0. Differing values can be used in alternate embodiments. 12 questions relevant to the force of Conversion and 12 more questions on Storage are also provided. The values of answers are added up for each force and are then normalized to a percentage value. For example, if the total value of Transport is 5, Conversion is 2 and Storage is 1

Ft=5/10*100=50

Fc=2/10*100=20

Fs=3/10*100=30

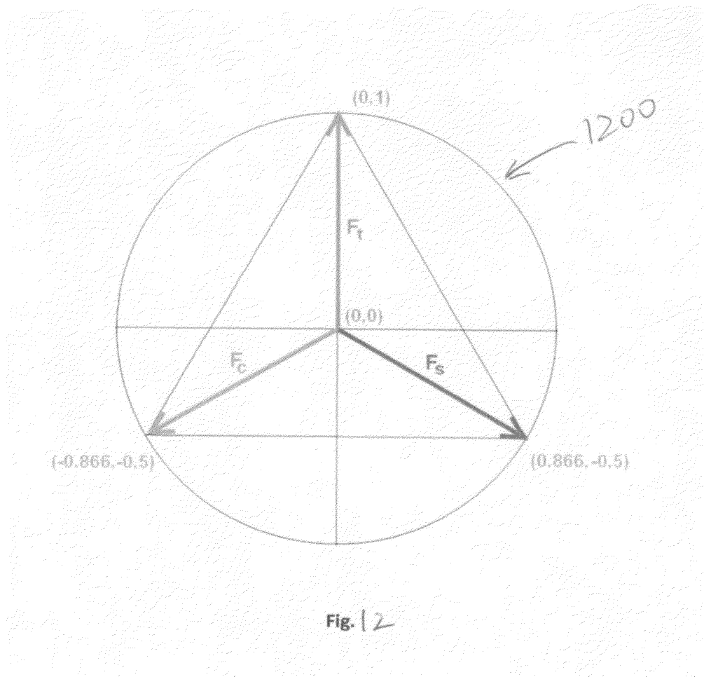

The following algorithm is then used to sum these three forces and represent the sum in a two-dimensional plane as depicted by the diagram 1200 of FIG. 12:

A circle is assumed with radius=1, centered at the origin.

A triangle is drawn such that the vertices are at the following location (0,1), (0.866, −0.5), (−0.866, −0.5).

Using vector algebra to represent the three Forces of Transport, Conversion and Storage:
Ft is a force that starts at origin and points to (0,1)
Fc is a force that starts at origin and points to (−0.866, −0.5), and
Fs is a force that starts at 0 and points to (0.866, −0.5).

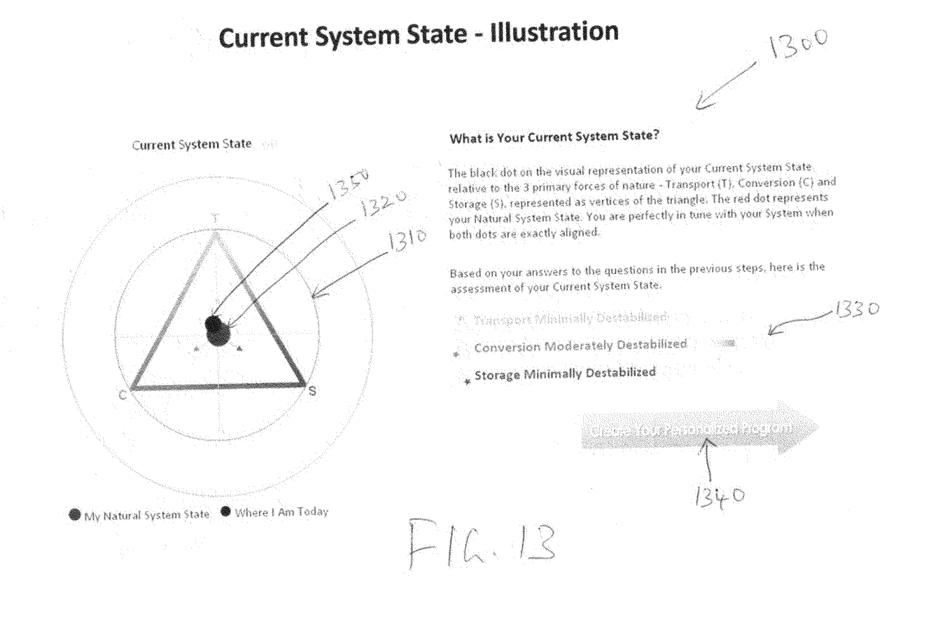

Once the Algorithm has plotted Current System State, the screen 1300 of FIG. 13 is presented to a user. A diagram 1310 shows a black dot 1350 for the user's Current System State, which is located off the red dot 1320 (Natural System State), depending upon which forces(s) is/are destabilized. The level of stabilization/destabilization is shown in region 1330 of the screen 1300. Based upon this displayed information, the user can click on the button 1340 create a personalized program aimed at improving stability of each force vector as appropriate.

Note that the three-dimensional forces listed are translated into a two-dimensional graphical representation along orthogonal X and Y axes using the following formula:

X=C*(0.866*(1+(Fs−Fc)))

Y=C*(1.5*Ft−0.5(Fs+Fc+1))

Note that this formula, and others described herein is illustrative of a wide range of mathematical operations used to compute various values and/or transform such values from one topology to another (e.g. from a three-dimensional representation to a two-dimensional analog).

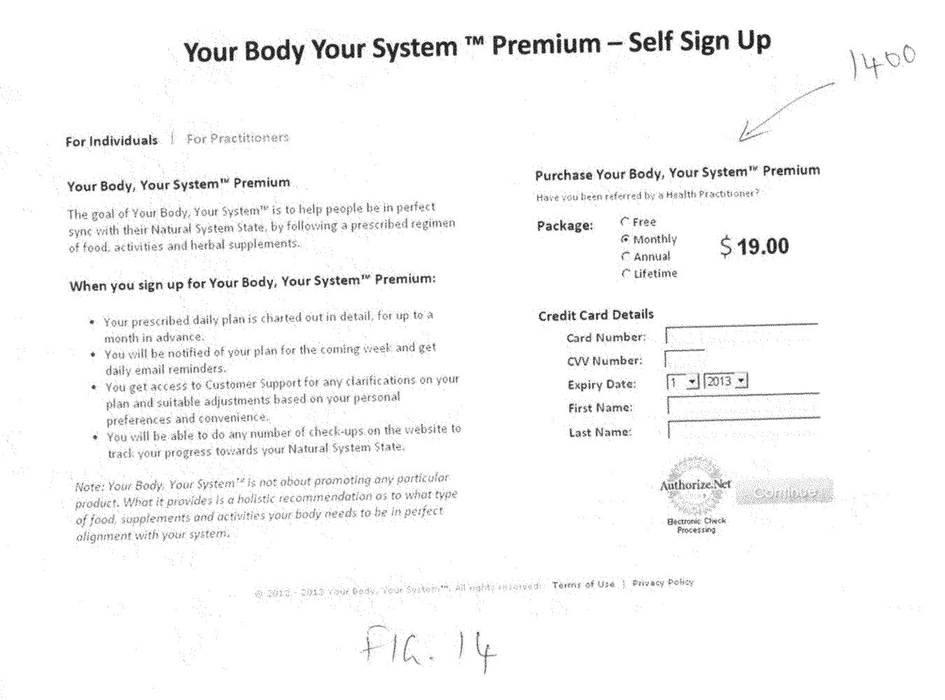

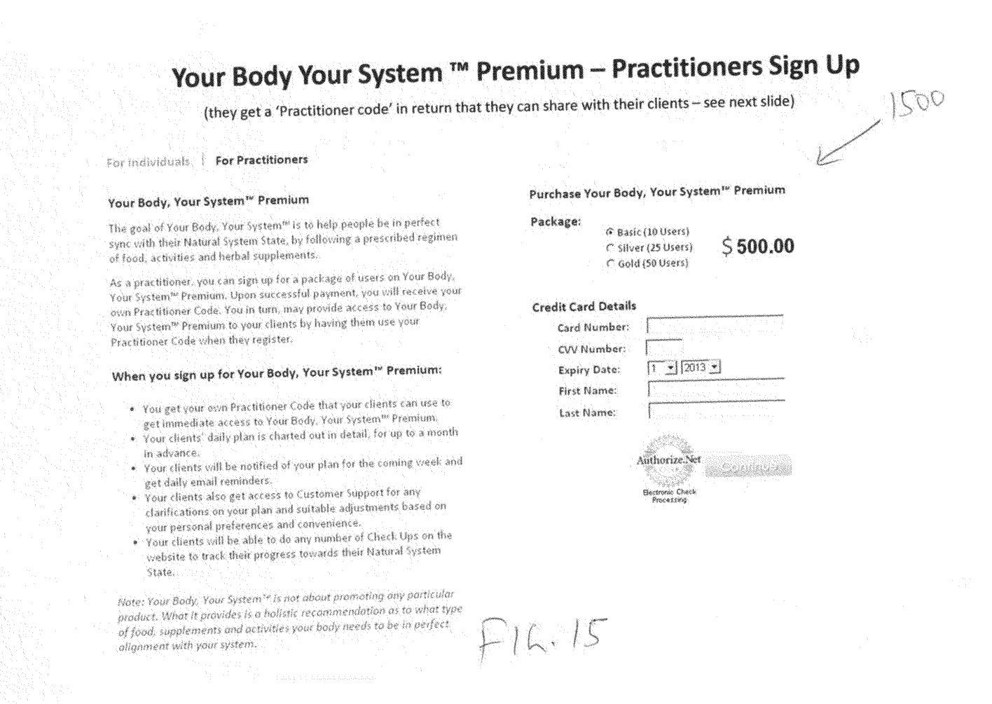

As part of the system's optional revenue model, a new user can be prompted to sign up (screen 1400 in FIG. 14) for a paid subscription service (e.g. a “premium service”) to obtain further functionality and access to system features. Similar sign-up screens (i.e. screen 1500 in FIG. 15 can be aimed at third parties, such as health and wellness professionals.

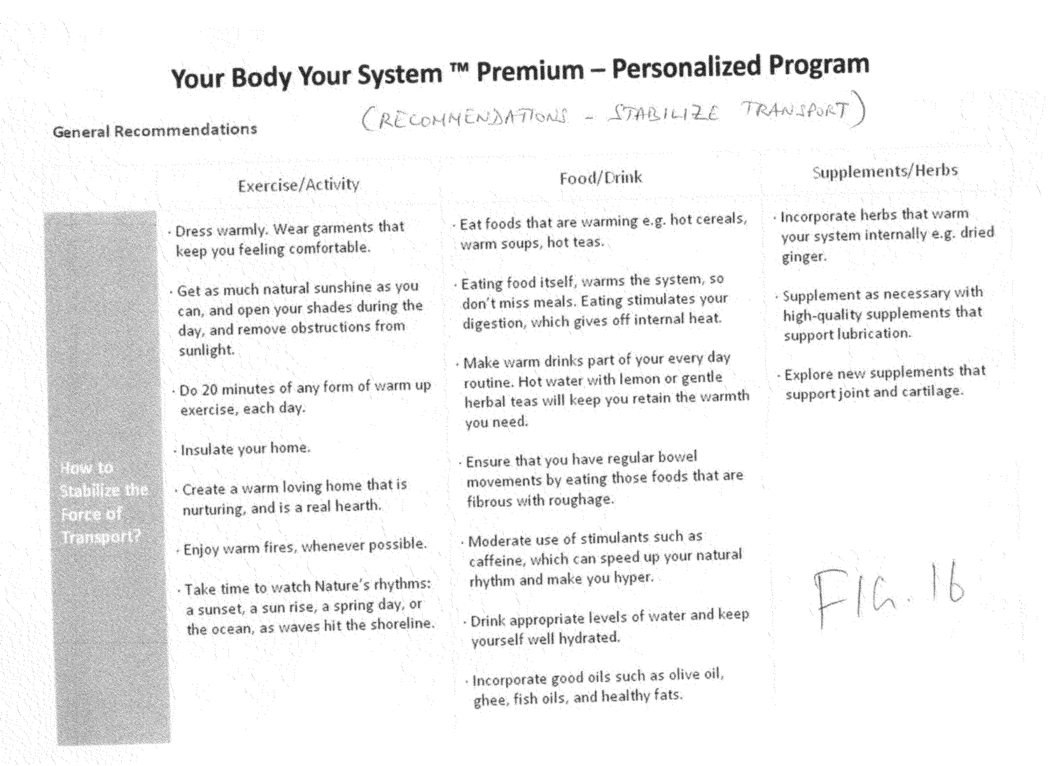

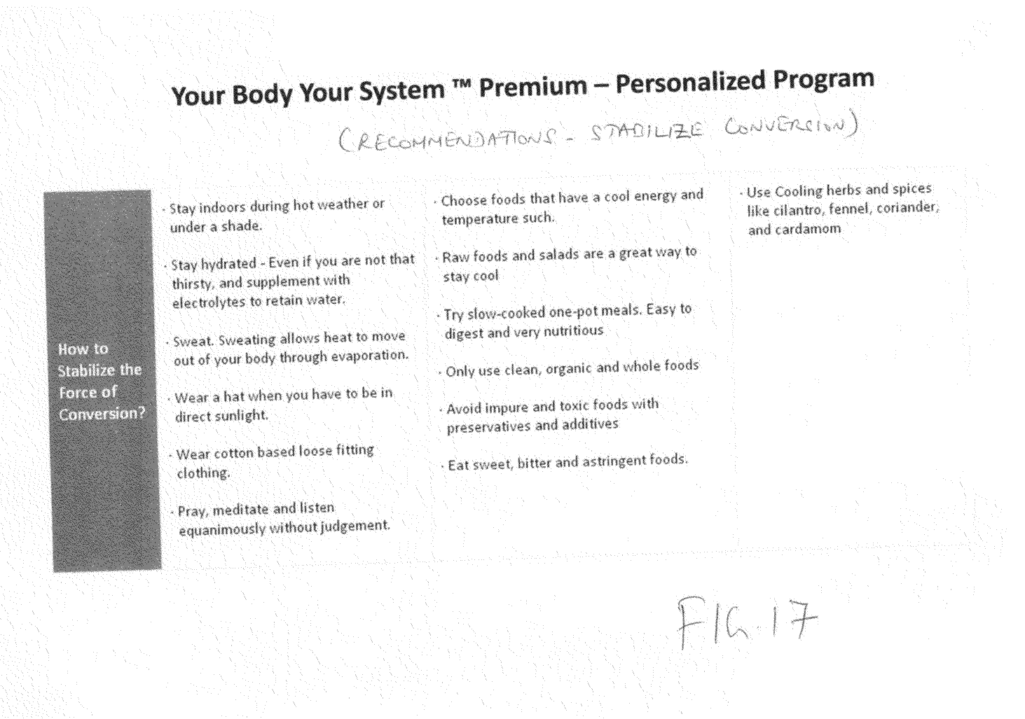

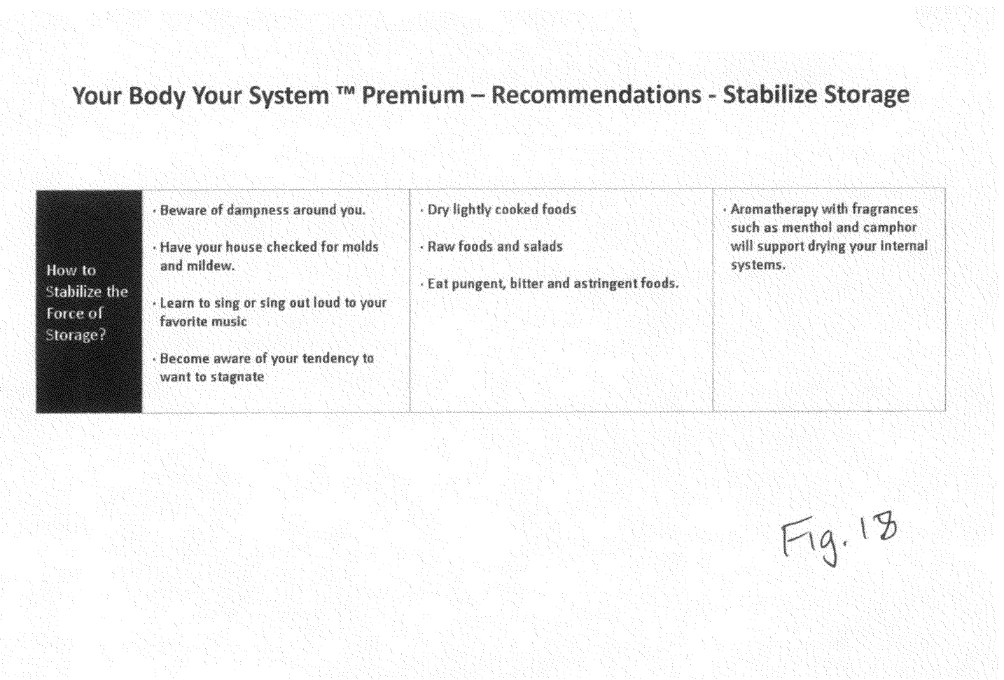

The difference between the user's Natural System State and Current System State is then assessed by the process, and using a database (stored and/or accessed through the Internet) of the relative impact of different foods/activities/lifestyle changes on the three forces of Transport, Conversion and Storage, a set of recommendations is arrived at. These recommendations are separated in screens 1600, 1700 and 1800 (FIGS. 16, 17 and 18) for each of Transport, Conversion and Storage vectors, respectively. Recommendations are provided in various categories as shown—for example relating to exercise/activity; food/drink, and consumption of supplements/herbs.

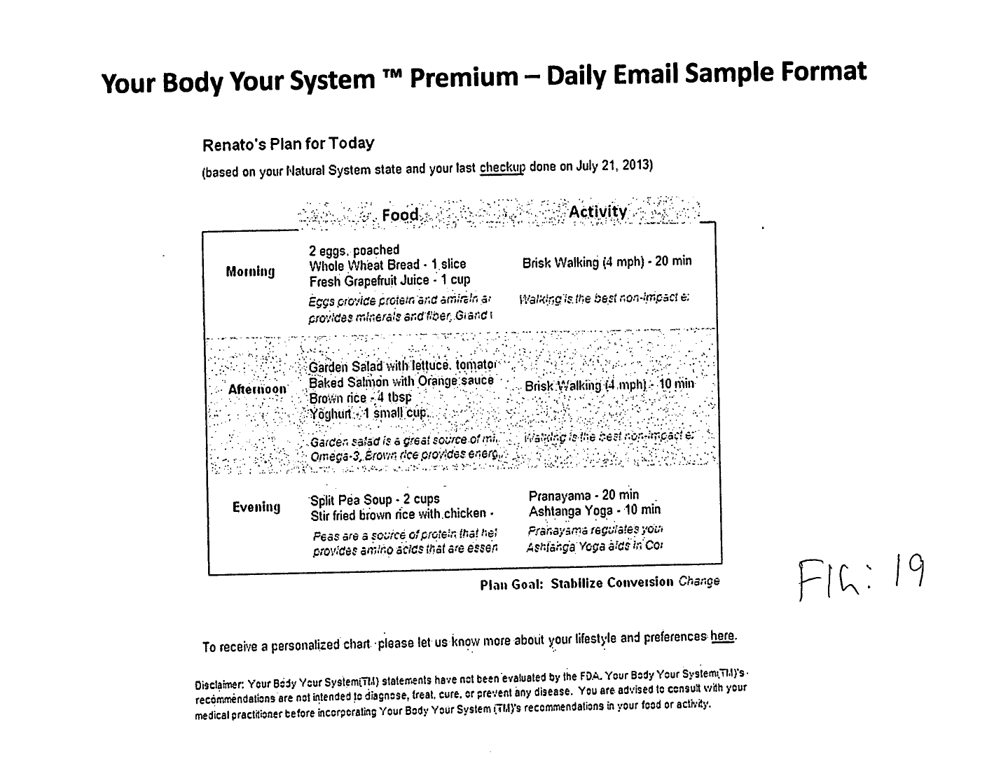

In FIG. 19, is an image 1900 of an e-mail received by a user. Notably, the user can customize delivery, format, mode of delivery (e.g. e-mail, text, etc.) of a reminder message. Such reminder messages can be (illustratively) sent to the user on a daily basis. Users can to perform repeated check-ups of their Current System State in the user interface to monitor improvement.

As described briefly above, the determination algorithm/process can be expanded to include certain biological and other relevant parameters/factors in connection with the user. Such factors can be entered via the user interface at the Natural System State phase or the Current System State phase of the overall process. FIG. 20 shows an illustrative interface screen 2000 that can be entered to provide biological parameters after (for example) deriving a base Current System state using the basic twelve questions for each of Transport, Conversion and Storage (see FIGS. 9, 10 and 11). This screen can reference existing, uploaded parameters 2010 and the resulting Current and Natural System State diagram 2020. The assessment of each system state parameter is provided at region 2030 of the screen 2000.

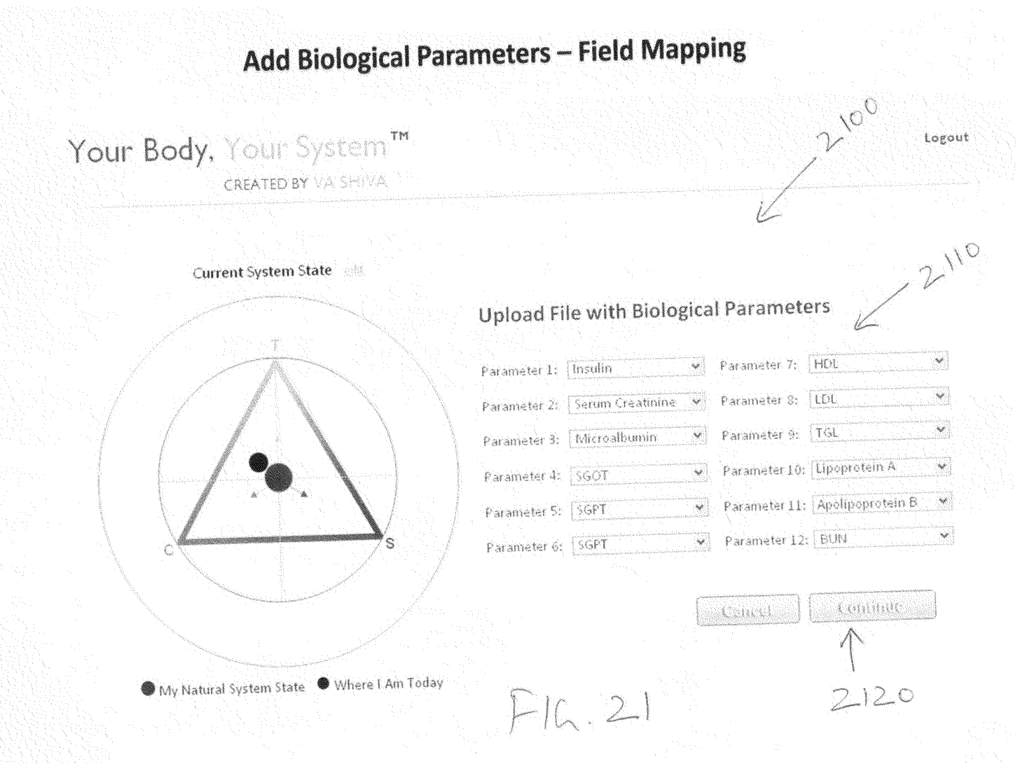

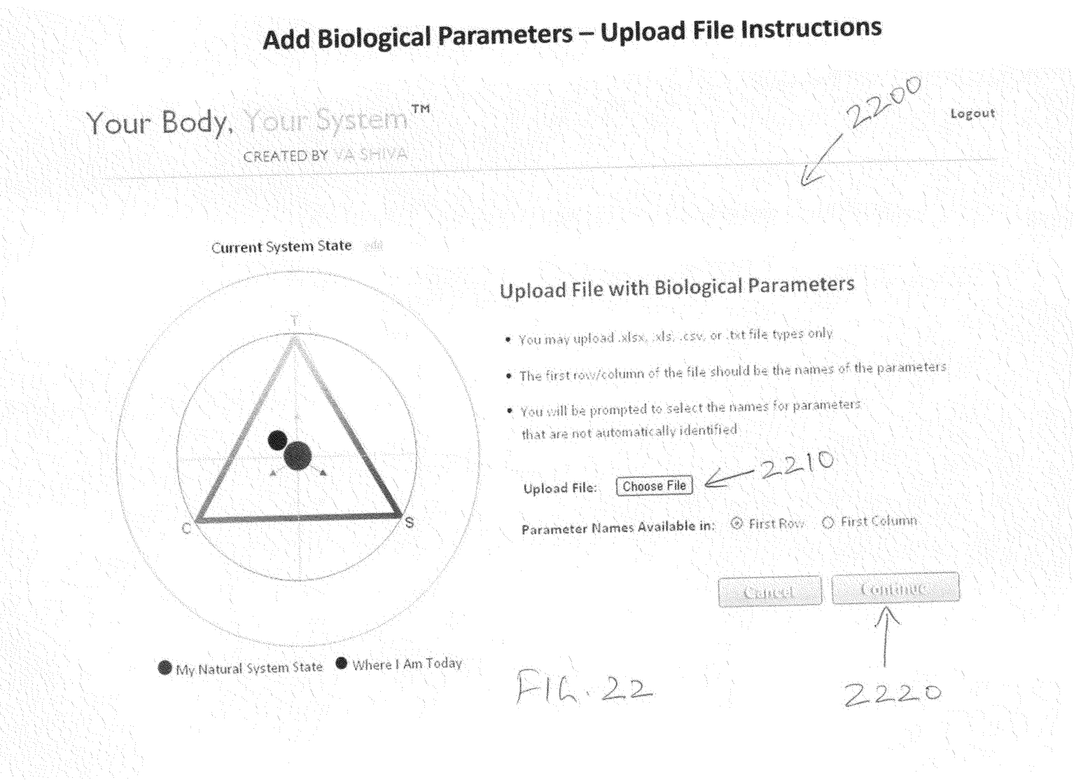

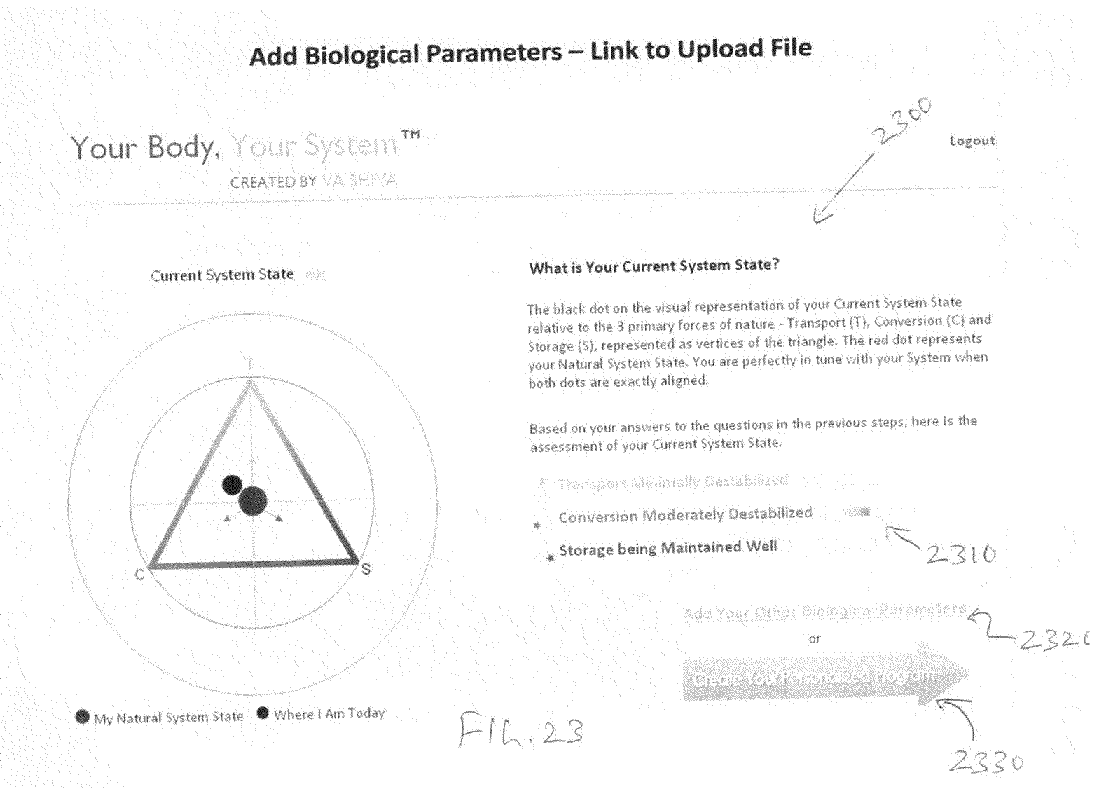

The button 2040 on this screen 2000 can be used to call up a follow-on screen 2100 shown in FIG. 21. This screen includes a serious of dialog boxes 2110 with associated drop-down menus for entry of various parameters, such as 9 but not limited to, insulin level, HDL level, LDL level, etc., as shown. Button 2120 allows the user to continue after identifying parameters to be provided via the boxes by accessing screen 2200 in FIG. 22. This screen allows such parameters (and associated values thereof) to be uploaded in one or more selected formats. The file can be accessed by browsing to an appropriate location on a computer memory structure using conventional techniques. After uploading the file using the upload file box 2210, the user then continues via button 2220 to screen 2300, which appears after the algorithm/process has included the biological parameters iii the Overall process—for example reweighting each vector based upon the effect of such parameters on it. The new, updated assessment of each vector is shown in region 2310. Note that, based on the exemplary uploaded biological parameters, the users vectors have been reassessed versus the initial assessment shown in region 2030 in FIG. 20. By way of example, Transport has been reevaluated from significantly to minimally destabilized based upon the added weight of the biological parameters. It should be noted that a variety of mathematical operations can be use to apply weightings and/or account for such parameters in a manner clear to those of skill. In the screen 2300 of FIG. 23, the user can subsequently add more parameters via the screens 2100 and 2200 by accessing button 2320 or continue to create a personalized program for improving well being via button 2330.

As noted, the above-described screens 2000, 2100, 2200 and 2300 can also be provided with appropriate modifications during the Natural System State determination phase of the process, and associated biological (and other related information) parameters can be entered to assist in determining the user's Natural System State.

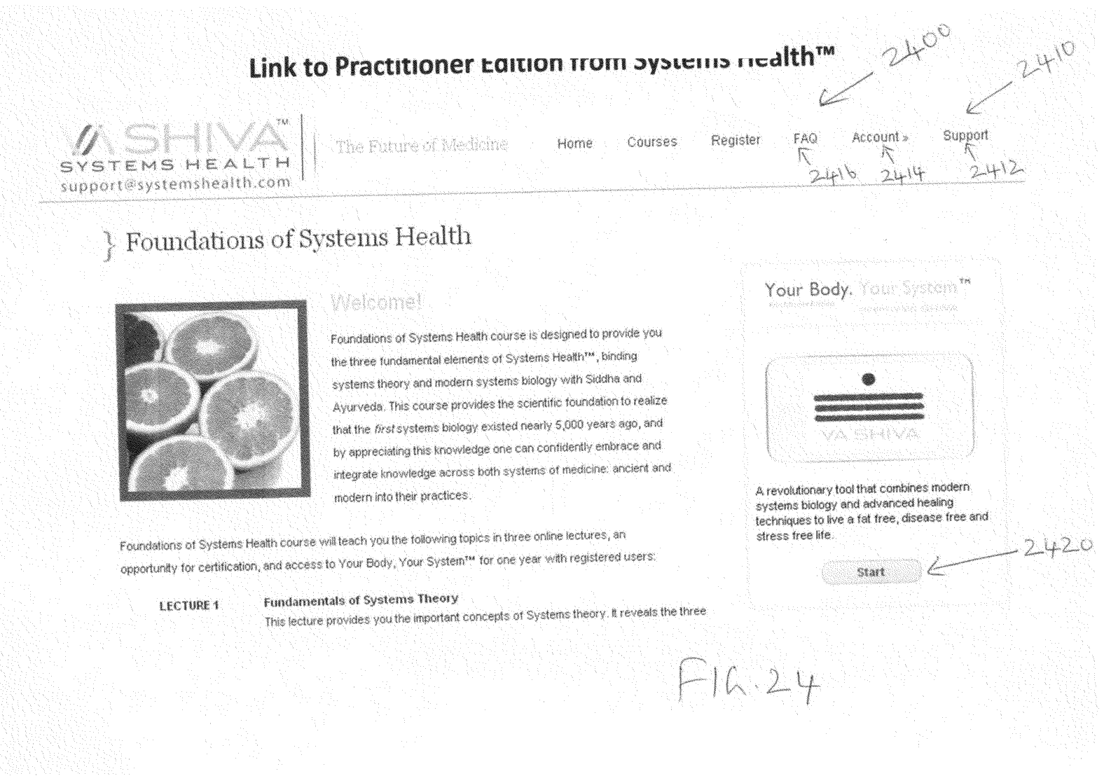

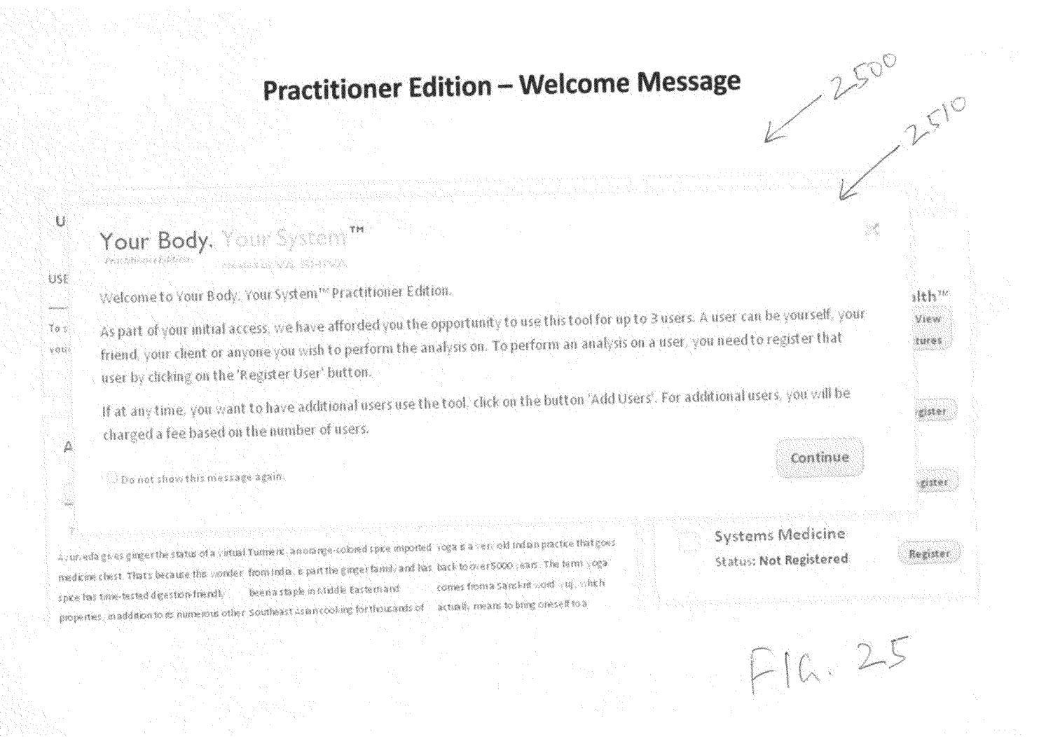

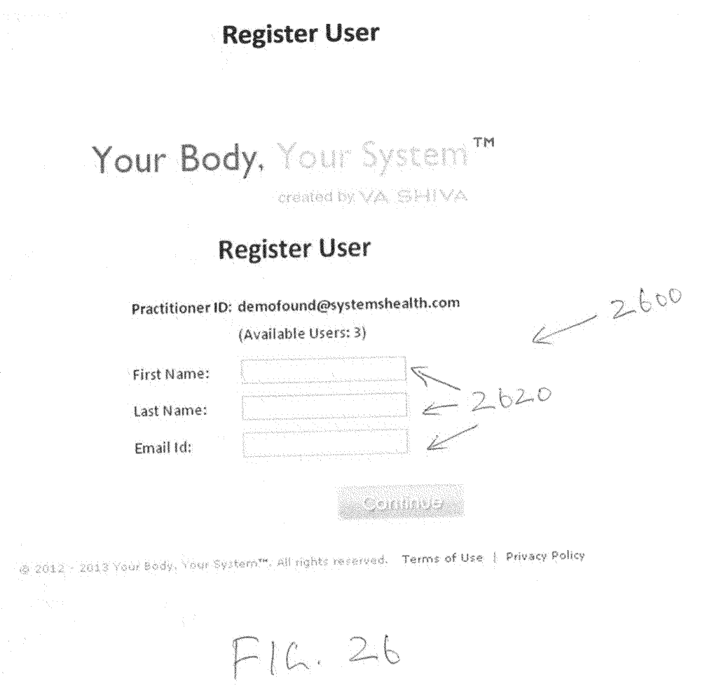

It is contemplated that various end-users of the system and method according to embodiments herein can lack sufficient skill or knowledge in handling the various queries and/or requests for information, such as biological parameters. Likewise, it can be desirable to afford various experts (teachers, medical professionals, care-givers, etc.) a vehicle to perform third-party analysis on clients/patients (users). For example, a yoga instructor can desire to perform an analysis on existing and new students. Thus, FIG. 24 shows a diagram of an optional user interface screen 2400 for a portal (Practitioner Portal) that provides third party practitioners with access to the illustrative system and method for use in analyzing system state of a patient/client according to an embodiment. After staring the process via tab 2420, the practitioner is presented with interface screen 2500 of FIG. 25, which provides a welcome page for the portal of FIG. 24 with a box 2510 with brief instructions on getting started with the Practitioner Portal. After entering the portal, a screen 2600 is provided as shown in FIG. 26. This screen 2600 contains a user registration/log-in dialog box arrangement 2620. After registering user information, the portal presents screen 2700, that provides information 2710 on the registered client/patient (user) for whom an analysis is to be performed by the system and method. In this example, the practitioner can track and analyze up to three users. In further versions (typically requiring a payment and/or subscription fee), the practitioner can register further users. The system tracks names and/or other identifying information, such as the user's e-mail and/or text address 2730. Once registration of the user is complete, the practitioner is presented with screen 2800 that invites him or her to enter the system for performing an analysis (check-up) on the registered client/patient (user). A button 2810 is clicked to begin the analysis. Screen 2900 is one of a plurality of screens presented to the practitioner as part of the system state analysis according to illustrative embodiments herein. As described above a series of questions/queries 2910 about the underlying user are presented in a yes/no format. The exemplary screen 2900 provides a series of questions/queries dealing with Transport for use in the determination process for establishing a user's Natural System State. Typically the practitioner can ask the user these questions orally and enter the appropriate information either contemporaneously with, or subsequent to, an interview. An arrow button 2920 allows the practitioner to navigate through further screens as appropriate (and as described above for the first-party version of the system and method) to answer questions/queries dealing with Conversion, Storage and entry of biological parameters for both Natural System State and Current System State. Results of analyses can be provided to the practitioner and/or user in a manner described above, using graphical and/or numerical displays.

It should be clear that the above-described system effectively captures, quantifies and allows a user to control key forces in their well-being. The delivery of information is intuitive and relatively straightforward to comprehend and act upon.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, a variety of graphical representations of the force Vectors can be employed in alternate embodiments. Where three dimensional displays are available, the subject matter can be displayed in such a format. Accordingly, this description is meant to be taken only by way of example, and not to otherwise limit the scope of this invention.

What is claimed is:

1. A system for determining a user's current system state comprising:
a determination process that computes force vector values based upon a plurality of questions and biological measurements provided via a user interface; and
a display generator that creates a representation of a location of current system state based upon the force vector values versus a location for natural system state.

2. The system as set forth in claim 1 wherein the representation defines a three-dimensional volume and the force vector values are arranged in three-dimensions therein.

3. The system as set forth in claim 2 wherein the Natural System State is located at a location point in the volume and the Current System State is located at another location in the volume.

4. The system as set forth in claim 2 wherein each of the force vector values is represented as a discrete color spectrum.

5. The system as set forth in claim 1 wherein the force vector values represent each of Transport, Conversion and Storage.

6. The system as set forth in claim 1 wherein users access the determination process through a client computing device and display interconnected through the Internet.

7. The system as set forth in claim 1 further comprising a user interface that is arranged to enable third-parties to provide information related to users for performing the determination process.
